(19) United States
(12) Patent Application Publication
(10) Pub. No.: US 2020/0105405 A1
(43) Pub. Date: Apr. 2, 2020

(54) DIFFERENTIAL DIAGNOSIS TOOL

(71) Applicant: Matthew Brandt, Springfield, MO (US)

(72) Inventor: Matthew Brandt, Springfield, MO (US)

(21) Appl. No.: 16/587,946

(22) Filed: Sep. 30, 2019

Related U.S. Application Data
(60) Provisional application No. 62/739,616, filed on Oct. 1, 2018.

(51) Int. Cl. G16H 40/20; G16H 50/20; G06Q 10/06

(52) U.S. Cl. CPC G16H 40/20; G06Q 10/06398; G16H 50/20

(57) ABSTRACT

The methods described herein include an improved method of training and assessing medical practitioners in the use of the differential diagnosis process. Risk and probability values assigned by a subject practitioner and a reference practitioner to a potential diagnosis for a patient are quantitatively compared to assess the performance of the subject practitioner in applying the differential diagnosis process. The method includes logically mapping the determinations of the practitioners to a data grid and calculating the sensitivity and specificity of the subject practitioner' determinations based on the data mapped onto the grid. The performance of the subject practitioner may be tracked to determine the amount of improvement over time.

### CROSS-REFERENCE TO RELATED APPLICATIONS

This application claims the benefit of U.S. Provisional Patent Application Ser. No. 62/739,616 filed on Oct. 1, 2018, the disclosure of which is incorporated herein by reference.

### BACKGROUND

### Field of the Invention

The disclosed invention is in the field of medical training and assessment systems and methods. More specifically, the disclosed invention is in the field of training student practitioners to perform diagnostic techniques such as the differential diagnosis method. The disclosed invention is also in the field of quantifying the difference in diagnostic determinations made by two or more practitioners with regards to a specific diagnosis. The disclosed invention is also in the field of evaluating the performance of a medical student or practitioner over time. The disclosed systems and methods effect various improvements in the technical field of medical training and evaluation, including the precise assessment and evaluation of a medical practitioner's performance in diagnosing medical conditions.

### Description of the Related Art

The use of the differential diagnosis process is well known in the practice of medicine. This process begins when a patient presents to a physician or other medical practitioner with a chief complaint. This chief complaint is related to a list of possible diagnoses that may be the cause of the complaint. The physician then utilizes deductive reasoning to eliminate diagnoses from the list of possible diagnoses based on information gathered from the patient and from the results of medical tests. The differential diagnosis process is iterative and continues until all potential diagnoses that may be eliminated based on available information have been eliminated from the list.

The efficiency and effectiveness of the differential diagnosis process depends on the skill and experience of the medical practitioner who is implementing the process. Training new medical practitioners to use the differential diagnosis process is difficult and time consuming for the teacher, preceptor, or reference practitioner. Training methods are not consistent within the medical community. Many medical practitioners-in-training are taught to use the differential diagnosis process solely by watching other medical practitioners use it, without any actual practice themselves or without useful feedback as to their success or failures, and without any measure of improvement over time.

Furthermore, there is no repeatable method of analyzing the use of the differential diagnosis process by more experienced medical practitioners. As medical practitioners continue through their career there is no objective way to measure and track their use of the differential diagnosis process to ensure that they are competent in their use of the process.

The inventive systems and methods for training and assessment described herein provide an objective and quantitative measurement of the difference between the diagnostic determinations of the trainer and the trainee, student and preceptor, or a reference standard and a medical practitioner, with respect to the differential diagnosis for a specific patient scenario. The objective and quantitative measurements may be tracked over time to identify trends in the performance of any given medical practitioner.

### SUMMARY OF THE INVENTION

In various embodiments, the inventive method described herein for assessing the performance of a medical practitioner in applying the differential diagnosis process to a patient having at least one potential diagnosis, consists of the following steps: receiving a first determination of a risk value and a probability value from the medical practitioner for the potential diagnosis, and receiving a second determination of a risk value and a probability value from a reference practitioner for the potential diagnosis, mapping the determinations to a logical data grid, calculating a performance value for the medical practitioner based on the cells in the logical data grid to which the first and second determination are mapped. The logical data grid comprises an array of cells having an origin, a first axis representing the risk value, and a second axis representing the probability value. In some embodiments of the method, the step of mapping the practitioner determinations to the logical data grid comprises selecting a set of cells in the data grid for each determination corresponding to the risk value and the probability value from the determination. The first set of selected cells may correspond to the determination made by the subject practitioner, and the second set of selected cells may correspond to the determination made by the reference practitioner.

In some embodiments of the inventive method, the performance value for the medical practitioner may include a specificity value. In some of these methods, the specificity value is calculated by calculating the number of true negative cells in the logical data grid, calculating a number of false positive cells in the logical data grid, and calculating the proportion of the number of true negative cells to the sum of the number of true negative cells and the number of false positive cells. The number of true negative cells in the logical data grid may be calculated as the number of cells in the logical data grid that are not in the first set of cells or the second set of cells. The number of false positive cells in the logical data grid may be calculated as the number of cells in the logical data grid that are in the first set of cells but not in the second set of cells.

In some embodiments of the method, the performance value for the medical practitioner may be a sensitivity value. In some of these methods, the sensitivity value is calculated by calculating a number of true positive cells in the logical data grid, calculating a number of false negative cells in the logical data grid, and calculating the proportion of the number of true positive cells to the sum of the number of true positive cells and the number of false negative cells. The number of true positive cells in the data grid may be calculated as the number of cells in the logical data grid that are in both the first set of cells and the second set of cells. The number of false negative cells in the logical data grid may be calculated as the number of cells in the logical data grid that are in the second set of cells and not in the first set of cells.

In some embodiments of the method, the steps of selecting a diagnosis, receiving a first determination, mapping the first determination, receiving a second determination, mapping the second determination, and calculating a performance value are repeated for a plurality of potential diagnoses. The performance value for each diagnosis in the plurality of potential diagnoses may be averaged to calculate a combined performance value for the medical practitioner. The performance value may be stored for the medical practitioner for a plurality of patients assessed by the medical practitioner over a period of time to evaluate a trend in the performance value for the medical practitioner.

In some embodiments of the inventive method, the process of selecting a set of cells in the logical data grid corresponding to a determination of risk and probability by a practitioner comprises selecting each cell in the logical data grid that has both (i) a risk value lower than or equal to the risk value from the practitioner's determination, and (ii) a probability value lower than or equal to the probability value from the practitioner's determination.

### BRIEF DESCRIPTION OF THE DRAWINGS

FIG. 1 is a flowchart of a method of using the differential diagnosis process.

FIG. 2 is a flowchart of a method of using the inventive systems described herein.

FIG. 3A is a depiction of an embodiment of a tool for implementing the described inventive systems and methods.

FIG. 3B is a depiction of an embodiment of a tool for implementing the described inventive systems and methods.

FIG. 3C is a depiction of an embodiment of a tool for implementing the described inventive systems and methods.

FIG. 3D is a depiction of an embodiment of a tool for implementing the described inventive systems and methods.

FIG. 3E is a depiction of an embodiment of a tool for implementing the described inventive systems and methods.

FIG. 3F is a depiction of a method of quantitatively analyzing data in an embodiment of the described inventive systems and methods.

FIG. 3G is a depiction of a method of quantitatively analyzing data in an embodiment of a tool for implementing the described inventive systems and methods.

FIG. 4A is a depiction of graphical user interface for an additional embodiment of a tool implementing the described inventive systems and methods.

FIG. 4B is a depiction of graphical user interface for an additional embodiment of a tool implementing the described inventive systems and methods.

FIG. 4C is a depiction of graphical user interface for an additional embodiment of a tool implementing the described inventive systems and methods.

FIG. 4D is a depiction of graphical user interface for an additional embodiment of a tool implementing the described inventive systems and methods.

FIG. 4E is a depiction of graphical user interface for an additional embodiment of a tool implementing the described inventive systems and methods.

FIG. 4F is a depiction of graphical user interface for an additional embodiment of a tool implementing the described inventive systems and methods.

FIG. 4G is a depiction of graphical user interface for an additional embodiment of a tool implementing the described inventive systems and methods.

### DETAILED DESCRIPTION

The differential diagnosis method is commonly taught to and used by doctors and other medical practitioners to evaluate a patient and determine a diagnosis for the complaints presented by the patient. For every complaint generated by a patient there is a lengthy but ultimately finite number of possible explanations for that complaint. The differential diagnosis process involves a determination of probability and risk for the various potential diagnoses that could be the cause of the complaint.

For each potential diagnosis the practitioner must assign a probability that the diagnosis applies to the patient based on the information at hand. The information may come from questioning, testing, or other investigations. Additional information subsequently leaves the probability of an individual explanation unchanged or reduces the possibility of that diagnosis. For example, for a patient with a chief complaint of abdominal pain a possible explanation could be ectopic, or “tubal” pregnancy. However, if we discover through investigation the patient is male that possible explanation is no longer applicable. Each question or examination posed by the practitioner changes the probability of a possible diagnosis. The medical practitioner utilizes a series of questions and observations to deductively eliminate possible but improbable diagnoses from that list of possible diagnoses generated by the chief complaint of the patient. The practitioner questions the patient, performs physical examinations and other medical tests, and takes other relevant steps generating objective data that ultimately renders some possible diagnoses improbable and, in some cases, removes some possible diagnoses from the list entirely until at some point a final list of possible diagnoses remains.

Each diagnosis has an inherent risk based on the potential outcomes for the patient if the condition is not identified and effectively addressed in a timely manner. For example, a diagnosis that will likely result in death if untreated is a “high” risk diagnosis. A diagnosis that will not result in death, but may result in harm if untreated, is a “moderate” risk diagnosis. Finally, a “low” risk diagnosis is one that will not result in harm to the patient if not immediately treated but will force the patient to seek additional care. Other categories of risk, with more or fewer gradations and differing names and definitions, may be utilized in various embodiments of the inventive systems and methods described herein.

The proper assignment of probability and risk is the key to the use of the differential diagnosis process. The ability of a medical practitioner, whether trainee or not, to assign appropriate values of probability and risk for each diagnosis for a patient has not been measured and analyzed in the past. The objective and quantitative analysis of a practitioner's ability to properly determine probability and risk as compared to a trainer or to a standard model is provided by the systems and methods described herein.

The deductive process is highly dependent upon the skill of the practitioner who is performing the process, therefore the process of training medical practitioners how to properly use the process is critical to the success of the treatment received by a patient. Initial training and assessment of medical practitioners in this process is often ineffective. It lacks a structured method for capturing the practitioner's use of the process, measuring the effectiveness of a particular practitioner's results, and tracking of the improvement of a practitioner over time. In most cases, the trainee's competence is assessed based on the subjective opinion of the trainer formed by observing or questioning the trainee. Similarly, the use of the differential diagnosis process by more experienced medical practitioners is also not assessed and tracked in a meaningful way throughout their careers.

In current medical training, the practitioner trainee's training is largely dependent on the practitioner trainer who may or may not (i) explain their use of the process on a patient to the trainee, (ii) determine how the trainee has used the process on a patient, or (iii) provide feedback to the trainee regarding how the trainee did and how the trainee may improve their use of the process. Furthermore, current training methods do not provide a structured way to allow a practitioner trainee to apply the process independently of the trainer to test the trainee's capabilities.

The invention disclosed herein provides structured systems and methods for measuring the use of the process by a practitioner trainee for a specific patient, comparing it to the use of the process by the practitioner trainer on the same patient, quantitatively analyzing the performance of the trainee, and measuring the change in a trainee's performance over time. Similarly, this process can be used to track the performance of a medical practitioner over time by comparing their performance as measured by the inventive systems and methods to standard procedures that may be established by the medical community. The standard procedures may be referred to as a “reference practitioner” that represents a reference determination set that may be used to test a medical practitioner.

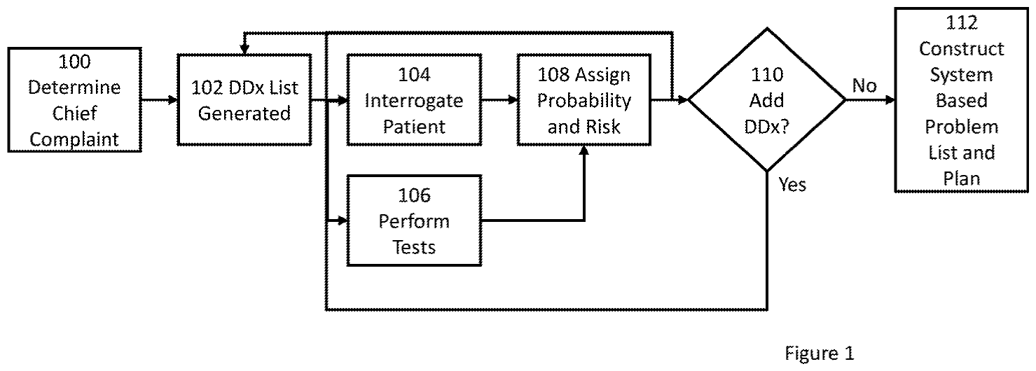

Referring now to FIG. 1, the typical differential diagnosis process begins with the identification of a chief medical complaint at step 100. The chief complaint is typically identified by the patient and is typically related to the symptom suffered by the patient without regard to the ultimate cause of the symptom. It is a short description of the medical issue being experienced by the patient. An example of such a chief complaint is “abdominal pain”. In other cases, it may need to be determined by the medical practitioner, for example, if the patient is unconscious or non-responsive to questions from the practitioner.

The identification of a chief complaint generates a list of possible diagnoses known as the Differential Diagnosis List or DDx 102. For example, the list of possible diagnosis for abdominal pain may include, without limitation, the following possible diagnoses: Abdominal Aortic Aneurysm, Appendicitis, Celiac's Disease, Cholecystitis, Cholelithiasis, Cirrhosis, Colorectal Cancer, Crohn's Disease, Cystitis, Diverticulitis, Dyspepsia, Ectopic pregnancy, Endometriosis, Gastroenteritis, Hepatitis, Gastritis, Intestinal Obstruction, Irritable Bowel Syndrome, Ovarian Cyst, Pancreatitis, Peptic Ulcer Disease, Peritonitis, Pyelonephritis, Small Bowel Obstruction, and Ulcerative Colitis.

The practitioner then gathers information to evaluate the diagnoses on the list. This information may be gathered from the patient at step 104 via questioning or physical examination, based on the patient's medical chart, determined via additional medical testing at step 106, or other methods as applicable. For example, some of the foregoing diagnoses are applicable only to female patients, and thus for a male patient, the following diagnoses may be immediately eliminated: Ectopic pregnancy, Endometriosis, and Ovarian Cyst. Further questioning of the patient or additional medical testing may be necessary to remove some of the possible diagnoses from the list or to assign a low probability to the diagnosis.

Once the practitioner has gathered the available information at step 108 they assign a probability and a risk value to each diagnosis in list 102. In some cases, the practitioner may iterate at step 110 from step 108 back to step 102 to add an additional diagnosis to list 102, ask additional questions at step 104, or run additional tests 106. The entire process is iterative and may continue until the practitioner feels that the process has sufficiently completed to make a final determination of risk and probability for each diagnosis. Once all the determinations of risk and probability are assigned to each diagnosis, a problem list and treatment plan may be created at step 112 to structure the treatment of the patient.

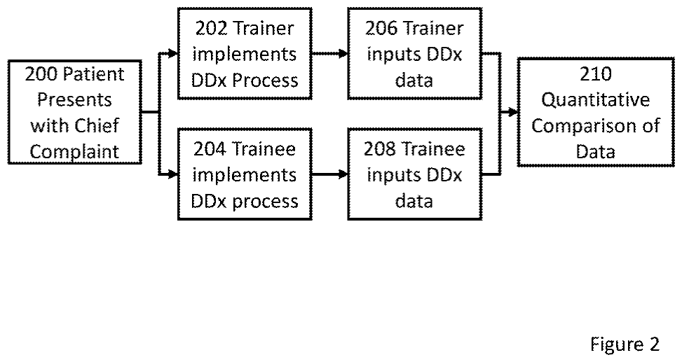

Referring now to FIG. 2, a flowchart of the use of an embodiment of the inventive systems and methods is depicted. The inventive systems and methods result in more efficient and effective training, evaluation, and assessment of practitioners who use the differential diagnosis method. In the depicted embodiment, a trainer and a trainee, such as an attending physician and a resident student, both independently apply the differential diagnosis process to a patient scenario. In some situations, the patient scenario may be provided as a case study. In preferred embodiments of the system, the patient scenario is an actual patient receiving treatment from an attending physician. The trainer may also be referred to as a reference practitioner, and the trainee as a subject practitioner.

The training and assessment process begins with the assignment of the chief complaint at step 200. The trainer and the trainee may independently determine the chief complaint which are NOT necessarily identical as two DDX lists may have several common explanations. As training progresses the trainer and trainee may independently determine the chief complaint.

Once the chief complaint is determined, the trainer and the trainee both implement the DDx process at steps 202 and 204, respectively. In some uses of the inventive systems and methods, the trainee may observe the trainer interrogating the patient, and may review medical test results and other information together with the trainer. In some embodiments, the trainer and the trainee may apply the DDx process independently of each other, with each performing separate interrogation and examination of the patient, and separately reviewing test results.

The trainer and trainee then separately assign probability and risk values to each diagnosis in the DDx list at steps 206 and 208, respectively. In preferred embodiments of the system, the data is input through a software application, web-based interface, or other similar means of collecting data and storing it in a database for processing. In software application embodiments, of the inventive systems and methods the data collected from the users is stored in a database in a format accessible for later processing for quantitative analysis. The database may be any format or system utilized to store and retrieve data, and no specific database technology is limiting of the scope of the inventive systems and methods.

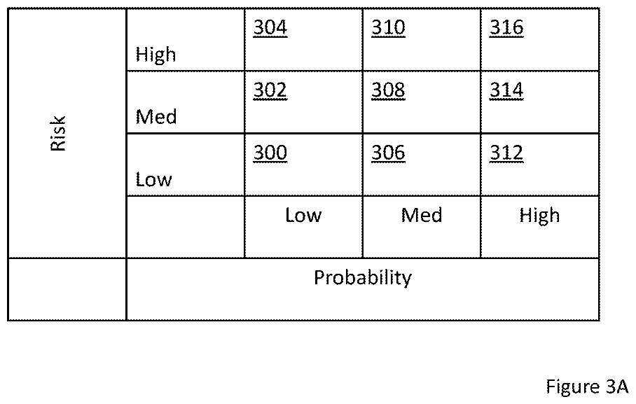

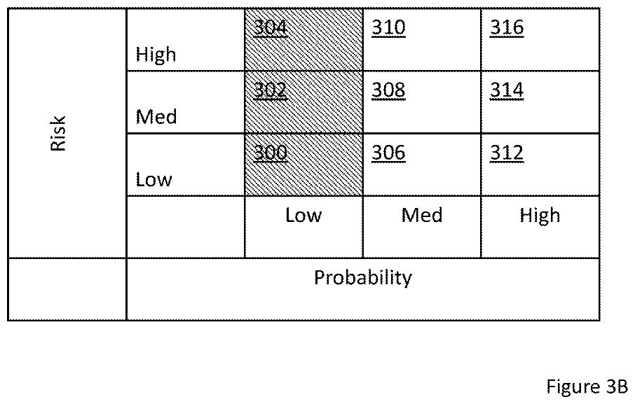

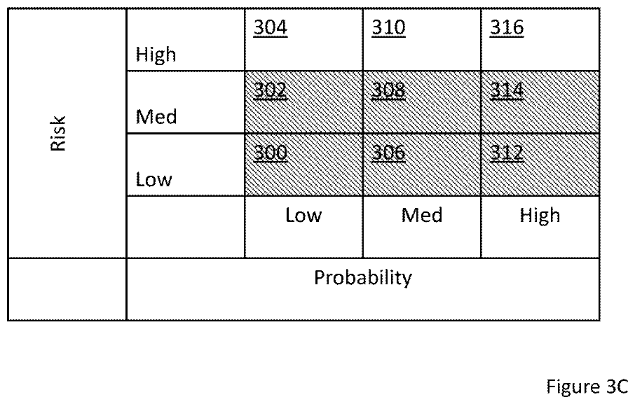

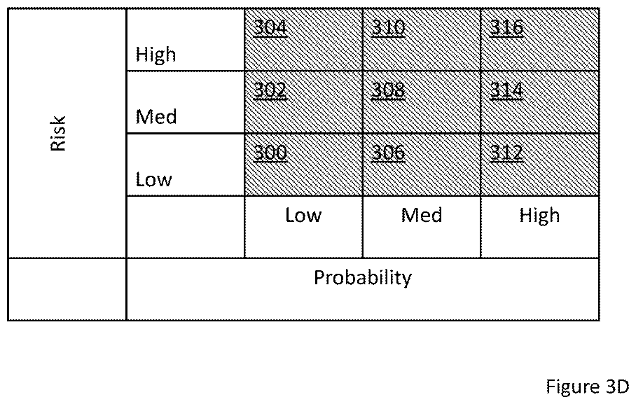

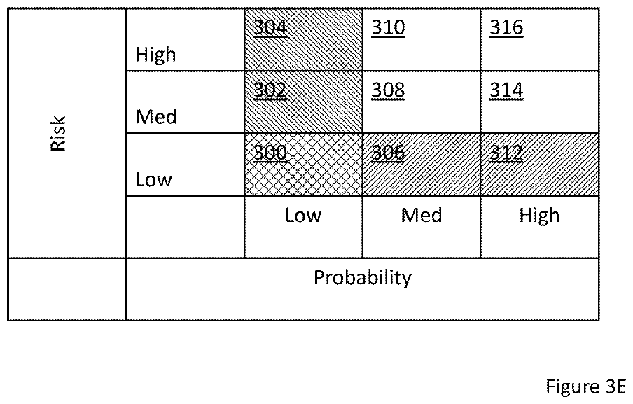

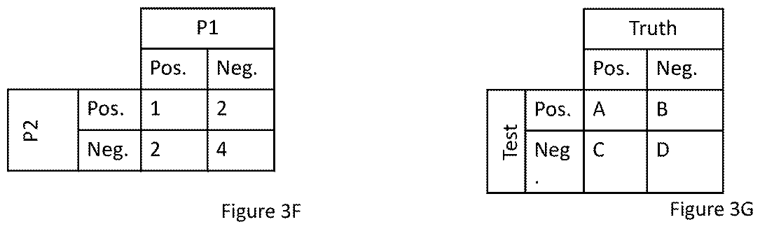

Referring now to FIGS. 3A-3E, an embodiment of an assessment tool for collecting data from a trainer and trainee, or other users, is depicted. The depicted embodiment may be collected on paper, but in a preferred embodiment of the system, the users input their determinations of the differential diagnosis into a computer application, such as a web-based application, a mobile-device based application, or similar applications. The specific technological method of receiving data from a user is not limiting of the invention, as those of skill in the art may implement the inventive system and methods using new computing and communications technologies as they are developed and made available for general purpose computing use.

The tool depicted in FIGS. 3A-3E allows a user to assign risk and probability values to a single diagnosis from the differential diagnosis list. The cells 300, 302, 304, 306, 308, 310, 312, 314, and 316 represent the product of potential risk and probability values that may be assigned to a specific possible diagnosis for a patient in one embodiment of the systems and methods. In other embodiments of the systems and methods, there may be more or fewer cells available depending on the gradations of risk and probability in that particular embodiment of the system and method.

In the depicted embodiment, the cells 300, 302, 304, 306, 308, 310, 312, 314, and 316 comprise a logical data grid with one logical axis that represents risk and another that represents probability. In this embodiment the “origin” of the data grid at cell 300 represents the “low” values for risk and probability. Cells farther away from cell 300 represent higher values for risk and probability. Each cell may be addressed as an ordered pair of values for risk and probability. For example, the cells in the data grid depicted in FIG. 3A may be addressed as (probability, risk) values such as (low, low) for cell 300, (medium, low) for cell 306, (high, high) for cell 316, etc. This allows the mapping of a pair of values for risk and probability onto the data grid. In other embodiments, the origin may represent the highest risk and other numbers of intermediate states between high and low may be potential values for the risk and probability determinations.

Mapping the risk and probability selections of the practitioners onto the data grid comprises selecting a set of cells in the logical data grid corresponding to a determination of risk and probability by a practitioner. In the preferred embodiment this mapping comprises selecting each cell in the logical data grid that has both (i) a risk value lower than or equal to the risk value from the practitioner's determination, and (ii) a probability value lower than or equal to the probability value from the practitioner's determination. This is different from selecting only a single cell that represents the exact value selected by the practitioner. It allows the comparison process to include the concept of the overlapping of the risk and probability selections of the two practitioners, not just whether they coincide exactly or not.

In other embodiments the number of risk levels may not by the same as the number of probability levels. The data grid is logical in the sense that it may be represented by a variety of different data structures, such as a pair of values as coordinates, an array of basic data types, an object coded in object-oriented coding languages, etc.

Although the user selects a single cell from the available options, the inventive systems and methods consider all cells that have the same or lower probability and risk as the user's choice as selected by the user. For example, if a user selected cell 304 as shown in FIG. 3B, the cells 300, 302, and 304 would be considered to have been selected for purposes of comparing the user's determination with those of another user. This selection corresponds to the shaded area in FIG. 3B which denotes a diagnosis with a high risk but low probability. For example, if a patient complains of chest pain, one of the differential diagnoses might include Acute Coronary Syndrome, or “heart attack.” Although the risk of this possible explanation is high as depicted we subsequently learn that the patient is 14 years old and thus the probability that this is the correct diagnosis may be determined to be low.

Similarly, if a user selects cell 314 as shown in FIG. 3C, then the inventive systems and methods treat all the cells shaded in that figure as selected, namely 300, 302, 306, 308, 312, and 314. This selection corresponds to a diagnosis with a medium risk and a high probability. A user selection of high risk and high probability results in the selection of all cells as shown in FIG. 3D.

Consider, for example, a patient with a chief complaint of chest pain. A possible DDx for chest pain is Acute Coronary Syndrome (ACS) or “heart attack.” However, subsequent questioning by the practitioner reveals that the patient is 30 years old, has no medical history of any kind, and his chest pain is better after taking antacids. A practitioner could conclude that although the risk of ACS is high, as missing this condition would result in death, the probability of this DDx being present is low because the patient is young and has findings more consistent with gastric reflux. Thus the practitioner's selection for the ACS diagnosis would resemble FIG. 3B. If, however, the patient is a 63-year-old male with a history of diabetes who experienced the acute onset of severe substernal chest pain radiating to this left arm and his EKG demonstrated findings consistent with an acute myocardial infarction the practitioner would rate the DDx of ACS high probability and the risk also high resulting in a risk probability chart as depicted in FIG. 3D.

Each user, for example, a trainer and trainee, independently input their determination of risk and probability for each diagnosis in the differential diagnosis list for a patient. Once all such determinations are collected by the system, the determinations for each diagnosis may be compared by the inventive systems and methods to determine the competence of the trainee. Referring now to FIG. 3E, an example is depicted in which a trainer has assigned a high risk/low probability to the diagnosis, while the trainee has assigned a low risk/high probability to the diagnosis. Thus for the trainer, cells 300, 302, and 304 are selected, while for the trainee, cells 300, 306, and 312 are selected.

With reference to FIGS. 3F and 3G, the two users are referred to as P1 for the trainer or reference practitioner, and P2 for the student, trainee, or assessment subject. For purposes of analyzing P2's determination in comparison to that of P1, the cells are categorized in 4 different types: (i) those selected only by P1 (“false negative cells”), (ii) those selected only by P2 (“false positive cells”), (iii) those selected by both P1 and P2 (“true positive cells”), and (iv) those selected by neither P1 nor P2 (“true negative cells”). This method of weighting and comparing the determinations made by the two practitioners is a novel way to translate the subjective determinations of risk and probability into quantitative data for comparison purposes. The comparison of the overlapping sets of cells onto which the determinations of risk and probability are mapped increase the accuracy of the assessment.

FIG. 3F depicts a graphical tabulation of the count of the 4 different types of cells as depicted in the assessment of the diagnosis depicted in FIG. 3E. In the example depicted in that figure, there are 2 cells selected only by P1, 2 cells only selected by P2, 1 cell selected by both P1 and P2, and 2 cells selected by P1 and not by P2. These counts quantitatively compare the selections made by the trainer and the trainee for that diagnosis. The counts are determined for each diagnosis in the diagnosis list.

The statistical concepts of sensitivity and specificity may then be determined for the comparison of P2 to P1 for each diagnosis. The system may thus utilize the data input by P1 and P2 to determine how “sensitive” and “specific” the determination by P2 is in relation to the determination of P1. The sensitivity of the determination may also be referred to the “true positive” rate and is the proportion of the “true positive” results to the total number of actual positive cases. The specificity of the determination may also be referred to as the “true negative” rate and is the proportion of “true negative” results to the total number of actual negative cases.

Referring to FIG. 9, a typical table for comparing the performance of a test to the truth of the situation is depicted. The upper left quadrant A represents “true positive” results. The upper right quadrant B represents “false positive” results. The lower left quadrant C represents “false negative” results. The lower right quadrant D represents “true negative” results.

In the inventive systems and methods, the sensitivity or “true positive” rate is calculated as the proportion between the number of cells that overlap between the determination of P1 and P2, corresponding to the upper left quadrant A, and the total number of positives identified by P1, corresponding to the sum of the upper left quadrant A and the lower left quadrant C, in FIGS. 8 and 9. The sensitivity value, as a percentage, is calculated using the formula A/(A+C).

Similarly, in the inventive systems and methods, the specificity or “true negative” rate is calculated as the proportion between the number of cells that were selected by neither P1 nor P2, corresponding to the lower right quadrant D, and the total number of negatives identified by P1, corresponding to the sum of the upper right quadrant B and the lower right quadrant D, in FIGS. 8 and 9. The specificity value, as a percentage, is calculated using the formula B/(B+D).

For the example depicted in FIG. 3F, P2 has a sensitivity of 1/(1+2)=33.3% and a specificity of 4/(2+4)=66.6% in relation to the determination of P1 for this diagnosis. Since a single chief medical complaint typically involves a number of individual diagnoses, the sensitivity and specificity values for a user P2 may be averaged across all diagnosis to determine an overall sensitivity and specificity. In any test or system evaluated using the sensitivity and specificity calculations, higher values for both numbers are indicative of better performance of the test, or in this case, of the performance of the practitioner in apply the differential diagnosis process to a particular patient.

In some embodiments of the inventive systems and methods, the sensitivity and specificity values for a particular user P1 and P2 may be stored in a database for further review and analysis. In some embodiments, a user P2 may be evaluated over time by the change in the sensitivity and specificity values calculated for such user. As a student proceeds through training the desired result is an increase in both sensitivity and specificity over time.

In embodiments of the system and method utilizing an application for data entry, the user may select a patient or case for which to enter data. In some embodiments, the user may be presented with the chief complaint which may have been assigned by the trainer or another user, while in other embodiments, each user may select their own chief complaint. Each user is then presented with a series of forms such as that depicted in FIG. 3A, one such form for each diagnosis in the differential diagnosis list.

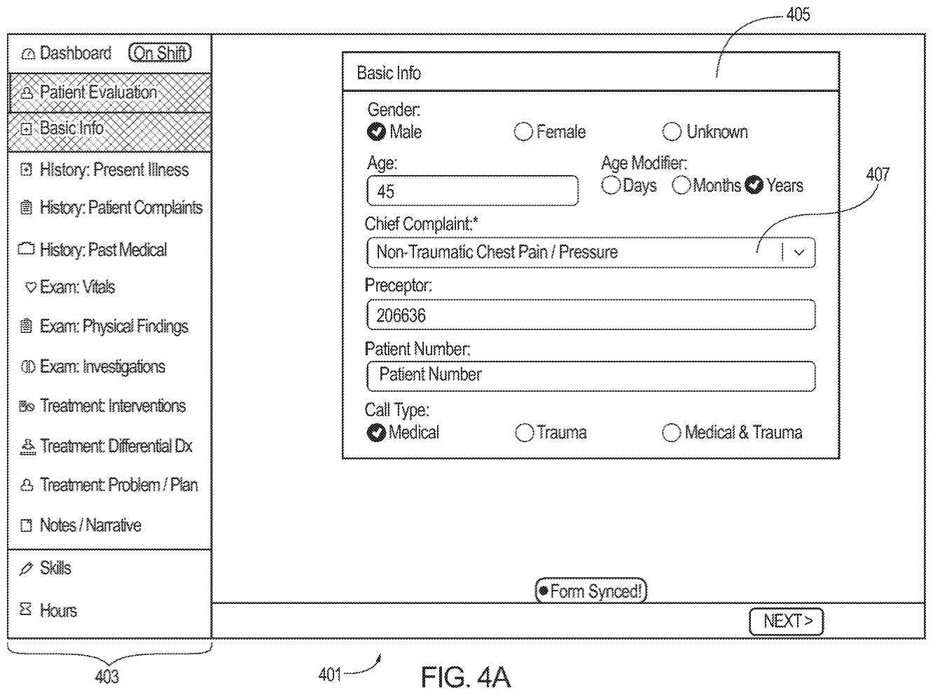

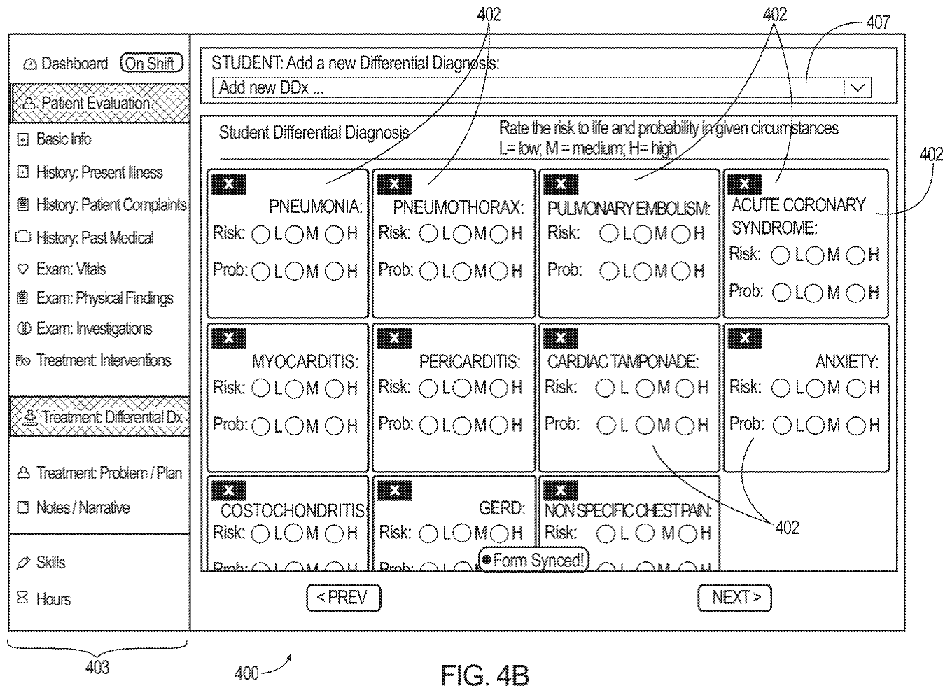

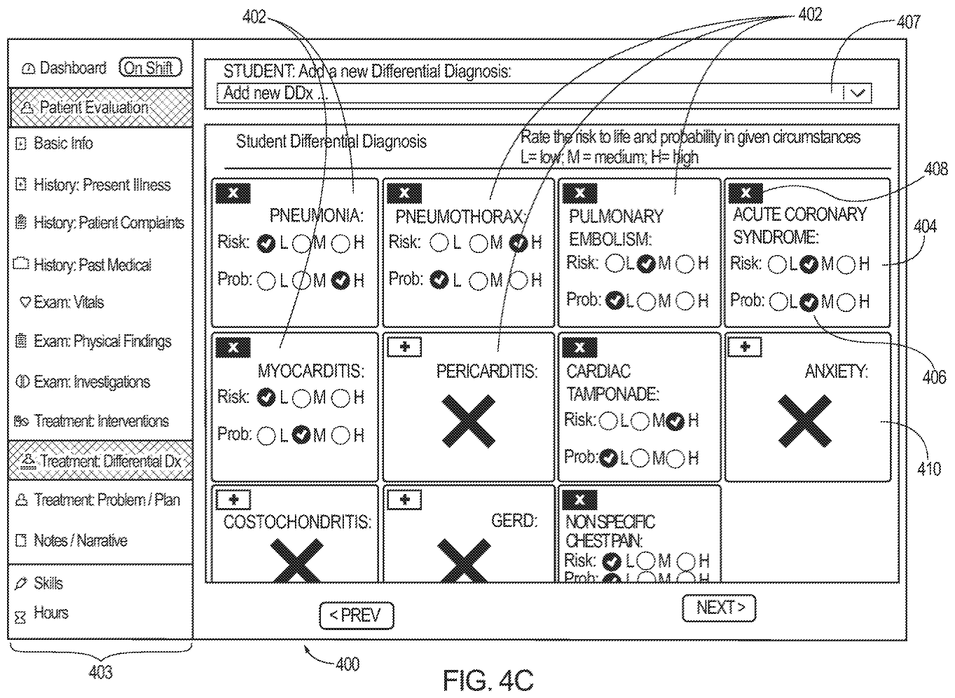

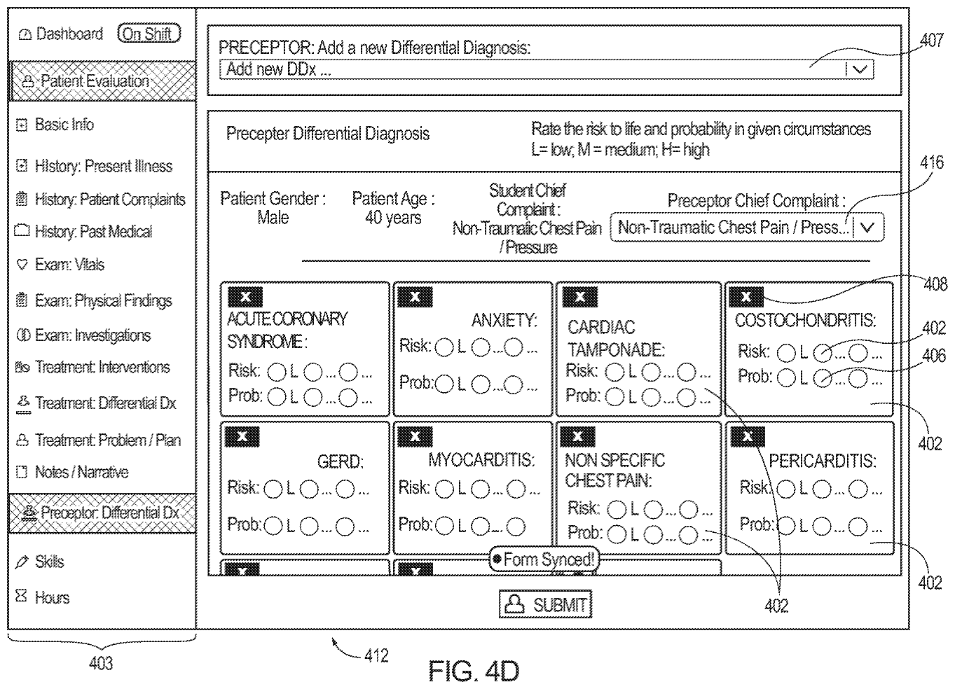

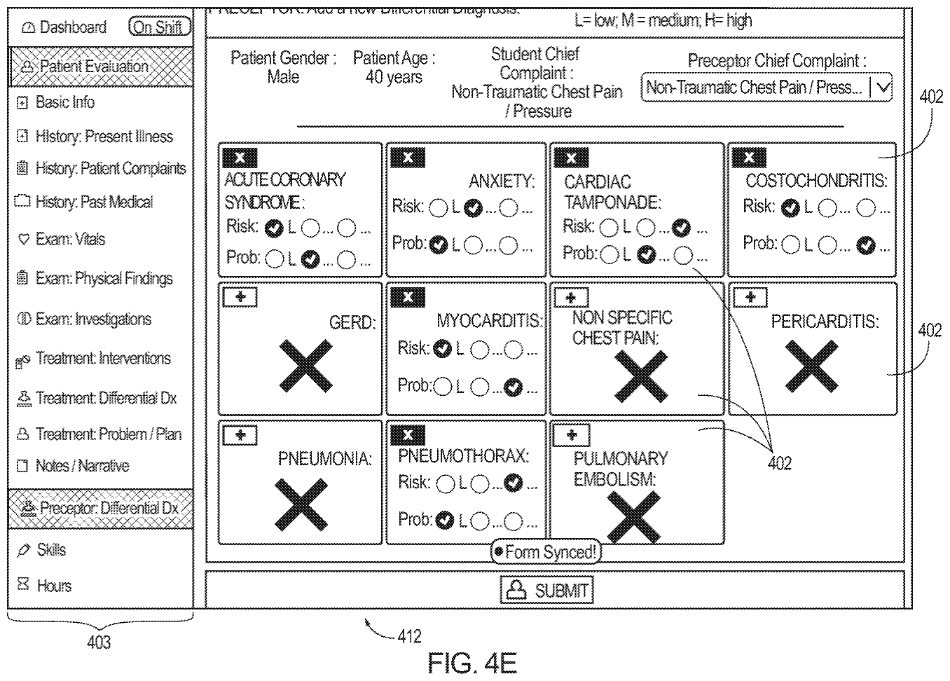

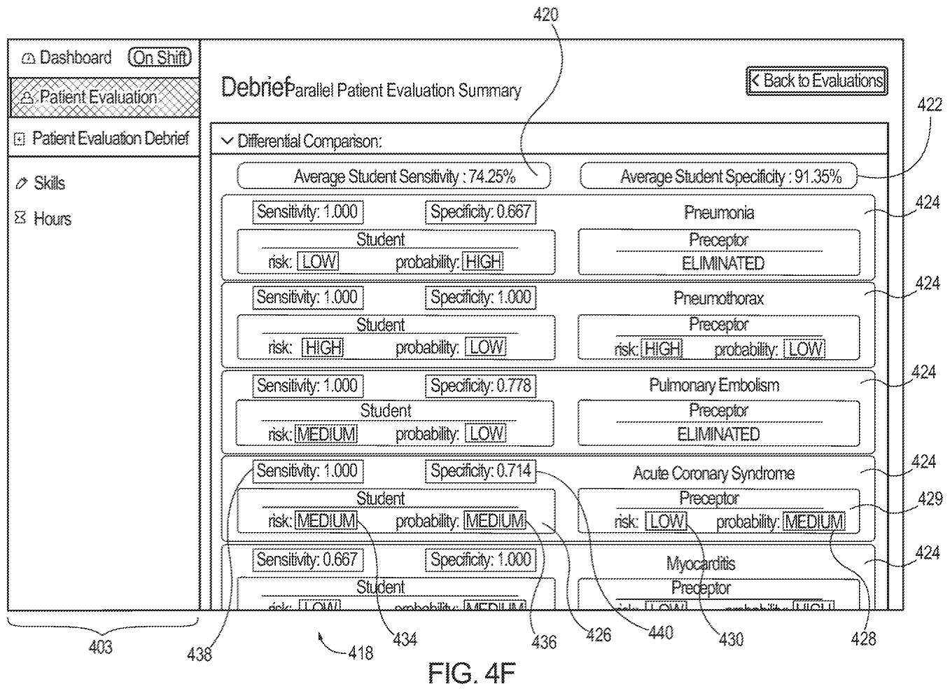

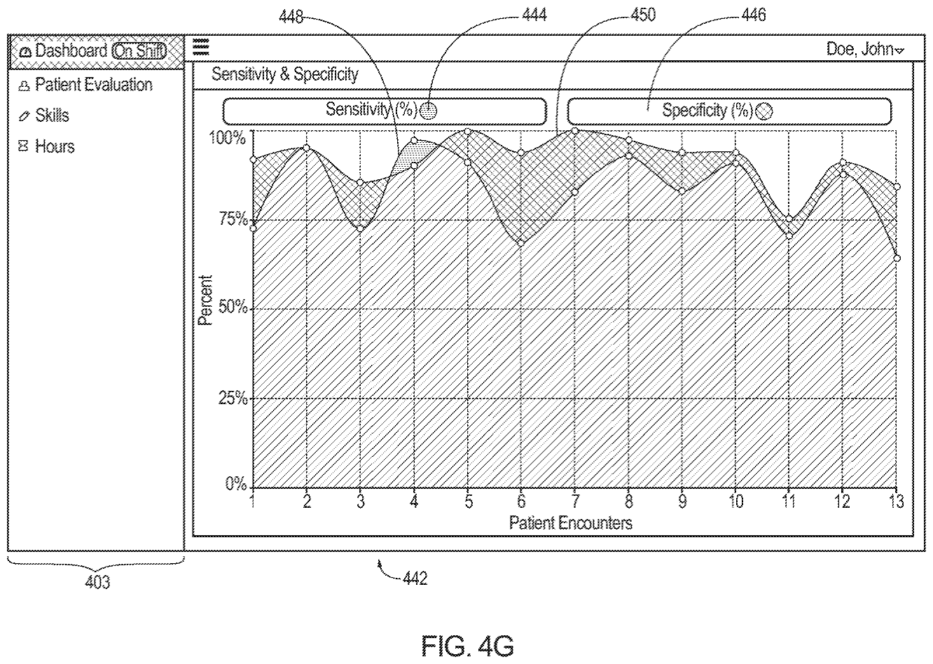

Referring now to FIGS. 4A-4G, various screens or interfaces are depicted from a graphical user interface for an embodiment of a system for implementing the inventive training methods. Many different graphical user interfaces may be utilized to collect, organize, and display the data and implement the methods described herein. These figures depict one of many embodiments that one of skill in the art of designing graphical user interfaces may create for this system and method. In some embodiments the system and method may be implemented using non-graphical text-based computer interfaces, a form-based interface, a natural language interface, voice command interface, or other similar interfaces developed for electronic devices.

In varying embodiments of the system and methods described herein, both a subject and reference practitioner may proceed through the process described in relation to FIGS. 4A-4G. In some cases, the subject is a medical student, EMT trainee, or other person receiving training in the differential diagnosis process. In other cases, the subject may be a practitioner who is being assessed for competence. In some cases, the reference practitioner may be a more senior medical practitioner, an attending physician, or a teacher. In other cases, the reference practitioner may comprise a dataset of expected or reference responses for certain patient examples.

Referring now to FIG. 4A, an embodiment of an input screen, or interface, 401 in an embodiment is depicted. In this embodiment one or more input interfaces may be provided to collect optional data about a patient. In screen 401 a sidebar 403 is provided with selectable tabs for various types of data to be collected about a patient, such as vital signs, test results, provider notes, and other information. These selectable tabs may also provide access to display interfaces that display historical information about a patient, and information about treatment plans and options. The sidebar 403 may also have direct data input controls in some embodiments. In other embodiments the sidebar 403 may not be present or may be replaced with a menu bar or tabs on the top of the screen. The data collection and display tabs are not required for all embodiments of the inventive system and method but may be useful in some embodiments to streamline the collection and review of data about a patient.

The depicted input screen 401 contains an input panel 405 for displaying graphical controls and input fields, such as gender, age data, and the Chief Complaint input control 407. In the depicted embodiment this is a “drop-down list”, but in varying embodiments it may be a free text field, a list box, a spinner, or a combination of the foregoing control elements. The subject utilizes input screen 401 to enter or select a value for the chief complaint presented by a patient. The chief complaint value entered by the subject may be stored in remote data storage such as a database, or it may be stored locally on the device on which the graphical user interface is displayed to the user. The specifics of data transfer, connectivity, and data storage not limiting of the scope of the inventive system and method. Some of the user interface screens may collect additional information that is not used in the inventive system. Once the subject has entered the chief complaint value, a button or other control may be used to move to a different set of control elements to input additional data. This may comprise clicking a button labeled “Next”, scrolling to a different section of the current screen, selecting a tab from sidebar 403, selecting a menu item from a drop-down menu, or pressing a key on a keyboard.

Referring now to FIGS. 4B and 4C, input interface or screen 400 is depicted for receiving information from the subject regarding the different potential diagnoses related to the selected chief complaint. The input panels 402 logically organize the input control elements manipulated by the subject to input data about each potential diagnosis. In this embodiment each diagnosis has a separate panel 402 on the interface 400. In other embodiments, the interface may comprise a data grid with a row for each diagnosis and controls in each row for the necessary input data values for each diagnosis. Similarly, other types of controls may be used to organize the input screen 400, such as a tab for each diagnosis, a selectable list of diagnosis that updates a single set of controls on the screen 400, or other interface styles that will occur to those in the art of interface design, all of which are within the scope of the inventive system and method.

In some embodiments of the system, the screen 400 may be populated with one or more diagnoses 402 based on the selected chief complaint. In other embodiments, the interface 400 may not have any preselected diagnoses 402 and the subject may have to select or enter each diagnosis from a control 407, which may be a drop-down list, a text field, or a combination of these or other types of control elements.

For each diagnosis 402, the subject is provided with control elements or input fields sufficient to allow input and collection of the data necessary to perform the inventive methods. These include a risk value and a probability value for each diagnosis. In some embodiments, this may also include an indicator of whether or not the diagnosis is applicable to the patient. In the depicted embodiment, the interface panel 402 for each diagnosis is provided with a control element 404 to accept the subject's determination of the risk associated with the diagnosis, a control element 406 to accept the subject's determination of the probability that the diagnosis is the cause of the chief complaint, and a control element 408 to indicate whether or not the diagnosis is applicable to the patient.

In the depicted embodiment, control elements 404 and 406 are “radio button” control elements that allow a user to select from one of a list of pre-defined alternatives. In this embodiment the options are “low”, “medium”, and “high” for both risk and probability. In other embodiments, these control elements may be drop-down lists or other control elements suitable for this type of data. In this embodiment, the control element is a button that may be depicted as an “x” or a “+”. If the diagnosis is currently indicated as applicable, the button will appear as an “x” and if clicked will change the status of the diagnosis to inapplicable and the panel 402 will alter to appear as shown in panel 410 which does not allow input of the risk and probability values. In that state, the button 408 changes to a “+” that when clicked will change the state back to applicable and the control elements 404 and 406 will reappear in panel 410. In other embodiments the button 408 may be replaced with a drop-down list or other control element, and there may be more than two states of the diagnosis.

Once the subject has entered all the diagnoses 402 via control 407, and then updated each panel for each diagnosis as shown in FIG. 4C, the subject may then submit the evaluation to the reference practitioner. In the depicted embodiment, that comprises selecting the “Next” button. In a preferred embodiment this step “locks” the subject's evaluation of the patient so that it cannot be changed or revised by the subject.

The reference practitioner then completes a similar process of entering a chief complaint and data regarding all the applicable diagnoses using an interface 412 such as that depicted on FIG. 4D. In this embodiment, the reference practitioner is presented with the subject's chief complaint but may select a different chief complaint using control element 416. The reference practitioner is also presented with the input panels 402 for each diagnosis. In this embodiment, the input panels 402 presented to the reference practitioner also have controls 404 and 406 to allow selection of “low”, “medium”, or “high” values for risk and probability, respectively. Once the reference practitioner has entered the data representing their analysis of the patient such as that depicted in FIG. 4E, they may submit the data by clicking the “Submit” button depicted on interface 412, or some other method as may be provided.

Once the reference practitioner submits their data to the system, it is processed using the inventive method as described above to evaluate the subject's performance in relation to the reference practitioner. Since the users input the data in this embodiment by selecting two values, one for risk and one for probability, those values may be converted into the logical equivalent of the selection of a cell depicted in FIGS. 3A-3E for purposes of quantifying the sensitivity and specificity for each diagnosis. For example, the selection of low probability and high risk shown for the “Cardiac Tamponade” diagnosis in FIG. 4C corresponds to the cell selection depicted in FIG. 3B. Each of the nine potential combinations of values for control 404 and control 406 correspond to one of the cells in the tool depicted in FIGS. 3A-3E.

A report or user interface summarizing the results, such as screen 418 depicted on FIG. 4F, may then be available to the subject and reference practitioner, as well as others who may be involved in monitoring and assessing the subject's training or performance. In the depicted embodiment, the report displayed on screen 418 includes a panel 424 for each diagnosis. In other embodiments, each diagnosis may have a row in a data grid, a line on a report, or other display options may be utilized to display the information. In a preferred embodiment, the report will display an average student sensitivity 420 and an average student specificity 422. In preferred embodiments, the risk value 430 and probability value 428 for each diagnosis from the reference practitioner will be displayed in the report such as in panel 429 on screen 418. Also, in preferred embodiments the report will contain the subject's analysis regarding each diagnosis including the subject's risk value 434 and probability value 436 such as depicted in panel 426. The calculated sensitivity 438 and specificity 440 for each diagnosis may be displayed in the report.

In some embodiments the results of each assessment may be stored in a database, data file, or other method of data storage. An interface may be provided such as interface 442 depicted in FIG. 4G, to allow the subject's performance to be monitored over time or over the number of patient encounters at the time of assessment. In a preferred embodiment the interface 442 displays the sensitivity 444 as graph 448 and specificity 446 and graph 450. In some embodiments it may be possible to sort and select data using additional criteria. For example, it might be desired to compare the application of differential diagnosis by multiple subjects for the same particular diagnosis, the performance of a subject on a specific type of chief complaint over time, or to track the progress of subjects who are taught by a specific reference practitioner or teacher over time to assess the performance of the teacher. These and other analytical uses of the data collected and created by the inventive method may be made by those in the field of teaching medical practitioners.

The data described in relation to the foregoing user interface may be organized in a different way on different screens and in a different order without departing from the scope of the inventive systems and methods.

Changes may be made in the above methods, devices and structures without departing from the scope hereof. Many different arrangements of the various components depicted, as well as components not shown, are possible without departing from the spirit and scope of the present invention. Embodiments of the present invention have been described with the intent to be illustrative and exemplary of the invention, rather than restrictive or limiting of the scope thereof. Alternative embodiments will become apparent to those skilled in the art that do not depart from its scope. Specific structural and functional details disclosed herein are not to be interpreted as limiting, but merely as a representative basis for teaching one of skill in the art to employ the present invention in any appropriately detailed structure. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

1. A method of assessing the performance of a medical practitioner in applying the differential diagnosis process to a patient having at least one potential diagnosis, the method comprising the steps of:
receiving a first determination of a risk value and a probability value from the medical practitioner assessing the at least one potential diagnosis with respect to the patient;
mapping the first determination to a cell in a logical data grid;
receiving a second determination of a risk value and a probability value from a reference practitioner assessing the at least one potential diagnosis with respect to the patient;
mapping the second determination to a cell in a logical data grid;
calculating a performance value for the medical practitioner based on the cells in the data grid to which the first and second determination are mapped;
wherein the logical data grid comprises a logical array of cells having a first axis representing the risk value and a second axis representing the probability value.

2. The method of claim 1 wherein the step of mapping the first determination comprises selecting a first set of cells in the logical data grid corresponding to the risk value and the probability value from the first determination, and the step of mapping the second determination comprises selecting a second set of cells in the logical data grid corresponding to the risk value and the probability value from the second determination.

3. The method of claim 2 wherein the step of calculating a performance value for the medical practitioner comprises calculating a specificity value.

4. The method of claim 3 wherein the specificity value is calculated by the steps of:
calculating a number of true negative cells in the logical data grid;
calculating a number of false positive cells in the logical data grid; and
calculating the proportion of the number of true negative cells to the sum of the number of true negative cells and the number of false positive cells.

5. The method of claim 4 wherein the number of true negative cells in the logical data grid is calculated as the number of cells in the data grid that are not in the first set of cells or the second set of cells.

6. The method of claim 5 wherein a number of false positive cells in the logical data grid is calculated as the number of cells in the data grid that are in the first set of cells but not in the second set of cells.

7. The method of claim 2 wherein the step of calculating a performance value for the medical practitioner comprises calculating a sensitivity value.

8. The method of claim 7 wherein the sensitivity value is calculated by the steps of:
calculating a number of true positive cells in the logical data grid;
calculating a number of false negative cells in the logical data grid; and
calculating the proportion of the number of true positive cells to the sum of the number of true positive cells and the number of false negative cells.

9. The method of claim 8 wherein the number of true positive cells in the logical data grid is calculated as the number of cells in the data grid that are in both the first set of cells and the second set of cells.

10. The method of claim 9 wherein a number of false negative cells in the logical data grid is calculated as the number of cells in the data grid that are in the second set of cells and not in the first set of cells.

11. The method of claim 2 wherein the steps of selecting a diagnosis, receiving a first determination, mapping the first determination, receiving a second determination, mapping the second determination, and calculating a performance value are repeated for a plurality of potential diagnoses.

12. The method of claim 11 wherein the performance value for each diagnosis in the plurality of potential diagnoses are averaged to calculate a combined performance value for the medical practitioner.

13. The method of claim 2 wherein the step of calculating a performance value for the medical practitioner comprises calculating a specificity value and a sensitivity value.

14. The method of claim 13 wherein the performance value is calculated for the medical practitioner for a plurality of patients assessed by the medical practitioner over a period of time to evaluate a trend in the performance value for the medical practitioner.

15. The method of claim 2 wherein the step of selecting a set of cells in the logical data grid corresponding to a determination comprises selecting each cell in the logical data grid that has both (i) a risk value lower than or equal to the risk value from the determination, and (ii) a probability value lower than or equal to the probability value from the determination.
